(19) United States
(12) Patent Application Publication
(10) Pub. No.: US 2004/0116499 A1
(43) Pub. Date: Jun. 17, 2004

(54) Indole derivatives having an inhibitory effect on protein kinases

(76) Inventors: Peter Mayser, Biebertal (DE); Wolfgang Steglich, Munchen (DE); Hans-Joachim Kramer, Butzbach (DE); Benhard Irlinger, Germering (DE)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/468,883

(22) PCT Filed: Feb. 26, 2002
(86) PCT No.: PCT/EP02/02024

(30) Foreign Application Priority Data
Feb. 26, 2001 (DE) 10109280.6

(51) Int. Cl. A61K 31/405

(52) U.S. Cl. 514/409; 514/414

(57) ABSTRACT

The invention relates to indole derivatives of formula (I) or (II) for use as inflammation modulators, especially having an inhibitory effect on protein kinases. Formula (I), wherein n=0 or 1, R¹=R²=3-indole, R³═COOH— or a ketone, X═O or NH; with the proviso that if n=0, R⁴═OH and R⁵=3-indole; and if n=1, R⁴ together with R⁵ represents an indole ring system condensed to the structure in the 2,3 position and X═NH. Formula (II), wherein R¹=R²=3-indole, X═O, CH₂ or a carbonyl group; X═O, a carbonyl group NH or >C═N(R′)₂ with R′═CH₃ or C₂H₅. The novel indole derivatives isolated from Melassezia are especially useful as substances for producing a medicament for the treatment of inflammatory or proliferative diseases, especially of the skin, but also of other organ systems.

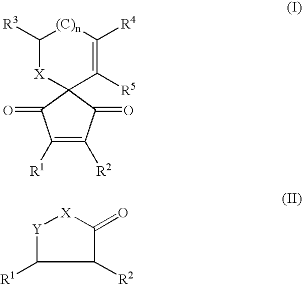

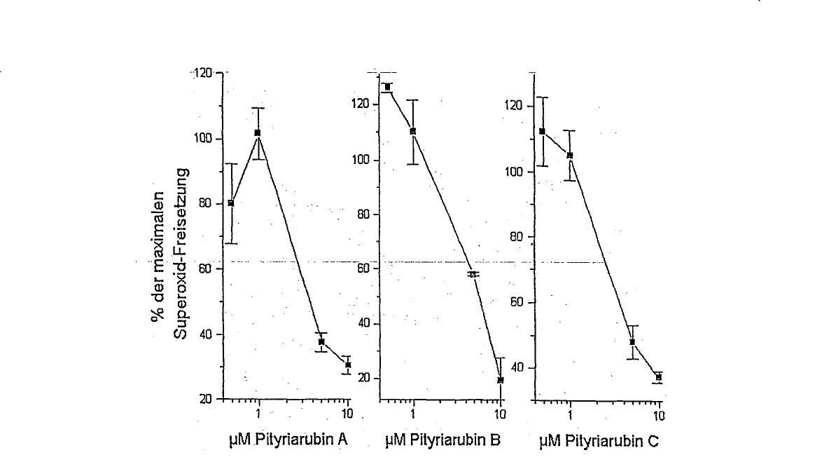

The present invention relates to novel indole derivatives having the distinct feature of efficiently inhibiting protein kinases as well as influencing other signal transduction processes involved in the neutrophilic burst. In particular, the invention relates to compounds which are suitable for the inhibition of protein kinase C (PKC) and its isoforms and/or which efficiently reduce the superoxide release of neutrophilic granulocytes. The derivatives according to the present invention are especially suitable for the use in the preparation of medicaments for the treatment of inflammatory and proliferative diseases, especially of the skin, but also of other organ systems.

Inflammatory and proliferative alterations of the skin and of other organ systems may represent an enormous medical, cosmetic and therapeutic problem. For example, the inflammatory skin diseases include psoriasis, eczemas, such as neurodermitis, the alterations caused by autoimmune processes, e.g. in Lichen ruber, Lupus erythematosus or (other) vasculitides, as well as all allergic processes caused by exogenic effects and skin alterations caused by infections. Similar alterations are found in inflammatory processes of inner organs caused by the respective noxes. To date, sepsis as a maximum form of inflammation represents a nearly unsolvable therapeutic problem. Furthermore important are diseases with an overactivation of inflammation cells, also e.g. within the scope of neoplastic and proliferative events, especially also of lymphocytes (parapsoriasis, mycosis fungoides, leucoses, lymphomas, pseudolymphomas) or in graft rejection.

According to the current prior art, protein kinase C dependent processes represent central control elements in the signal transduction of inflammatory processes, lymphocytic and granulocytic activation, cytokine release and antibody production. However, while protein kinase C inhibitors of the bis-indolyl maleimide type known from the prior art represent valuable tools for the investigation of PKC dependent processes, they did not enter into therapy due to their relatively low potency and specificity.

Thus, it is the object of the present invention to provide immune modulators, especially PKC inhibitors, which far exceed the inhibitors known from the prior art in their inhibitory effect and which are well suited for the preparation of tolerable medicaments for the treatment of a broad spectrum of disease related skin and organ alterations.

In the following, immune modulator means a substance which is able to activate or to inhibit the immune system or a portion thereof. For example, such substances may be inhibitors or activators of the formation and/or secretion of cytokines, leukotrienes, interleukins etc.

Substances, which are able to inhibit or activate cells of the immune system, e.g. T cells, β cells, dendritic cells, makliophages, neutrophiles, granulocytes etc. are also included within the definition of immune modulators. Assays are known to the person skilled in the art by means of which it may be determined whether a substance inhibits or activates the processes mentioned above. For example, the inhibition of the granulocyte burst is described further below as such a process for the identification of an immune modulator

Surprisingly, it has been found according to the present invention that a specific subpopulation of the yeast genus Malassezia, in particular the yeast species Malassezia furfur, is able to synthesize potent novel inhibitors of protein kinases, especially protein kinases C when particular nutrients are supplied. If Malassezia is supplied with the amino acid tryptophane (as L- or D-isomer or also as a racemate) as the predominant nitrogen source the active ingredients of the present invention may be isolated from a yeast conditioned in this manner. In particular, it has also been found that when tryptophane substituted at position 4, 5, 6 and/or 7 of the indole ring is supplied said substituents are incorporated into the indole derivatives which are synthesized by Malassezia and on which the present invention is based in a nearly unaltered manner. This is of particular interest if the relatively hydrophobic indole derivatives synthesized from pure tryptophan-e are to be made more hydrophilic, e.g. by means of hydroxy substituents.

The protein kinase inhibitors of the present invention show a particular specificity for T- and B cell specific PKC isoforms. Thus, they are particularly suited for the investigation of such processes and as immune modulatory, anti-inflammatory and anti-proliferative active ingredients. Furthermore, these PKC inhibitors surprisingly show an antibiotic effect against gram-positive bacteria, in particular also against multi-resistant staphylococci (MRSA).

Further, the substances described are able to inhibit the neutrophilic burst (as a model for inflammation processes), wherein also other signal transduction processes (including cytokine release and leukotriene synthesis) may be influenced in addition to PKC. In particular, this comprises the competitive binding to all kinds of receptors involved (also those unknown to date) with the result of a displacement of the physiological ligand to a net inhibition of the cellular event.

A portion of the compounds isolated from Malassezia with distinct protein kinase inhibitor action and distinct inhibitor effect on the cellular control events associated with the superoxide release of neutrophilic granulocytes are indole derivatives characterized by a spiro C atom and which have the following general structure:

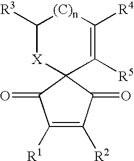

wherein:

n=0 or 1;

R═R²=3-indole;

R³═COOH or a ketone;

X═O or NH;

with the proviso that if n=0, R⁴═OH and R⁵=3-indole; and if n=1, then R⁴ taken together with R⁵ represents an indole ring system fused at position 2,3 and X═NH,

wherein the indole ring systems may be each substituted individually or in combination with substituents, selected from the group consisting of OH, F, Cl, Br, NO₂, NH₂, COOH, HSO₃ at position 4, 5, 6, and/or 7 or form aza compounds.

Spiro compounds having the following structures are preferred:

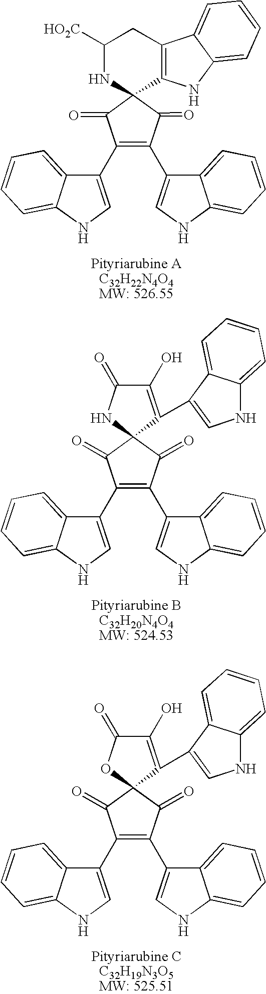

Pityriarubine A contains an asymmetric center. Both isomers as well as the racemate exhibit a distinct inhibitory effect.

The pityriarubines exhibit a similar structure as the known substance group of the bis-indolylmaleimides, however, they have a spiro C-atom instead of the amide nitrogen in the bis-maleimides. Thus, the pityriarubines represent another novel substance class and are also not simple derivatives of the bis-indolylmaleimides.

Further compounds isolated from Malassezia according to the present invention having a distinct inhibitory effect to PKC and the neutrophilic burst are not spiro compounds and are represented by the following general structure:

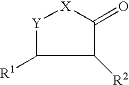

wherein:

R¹=R²=3-indole;

X═O, CH₂ or a carbonyl group;

X═O, a carbonyl group, NH or >C═N(R′)₂

wherein R′═CH₃ or C₂H₅;

wherein the indole ring systems may be each substituted individually or in combination with substituents, selected from the group consisting of OH, F, Cl, Br, NO₂, NH₂, COOH, HSO₃, at position 4, 5, 6 and/or 7 or form aza compounds.

Preferred representatives of compounds having said basic structure are:

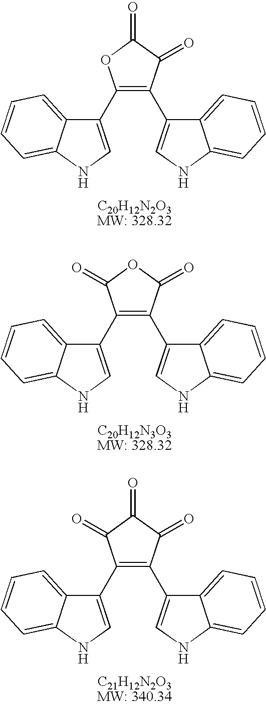

In these compounds the indole ring systems may also be substituted at position 4, 5, 6 and/or 7 each individually or in combination with the groups mentioned above.

The above compounds may be isolated according to the following method:

A yeast subpopulation of the genus Malassezia, in particular of the species Malassezia furfur is supplied with the amino acid tryptophane (L-, D-isomer or racemate) as the predominant or only nitrogen source. From the pigments and fluorochromes produced by Malassezia under said conditions the compounds mentioned above may be isolated.

As a suitable nutrient medium 30 ml Tween® 80 ultra (Sigma, St. Louis, USA) and 20 g Agar ultrapure (Merck), filled up to 1 l with water, are autoclaved. Following cooling to 50° C. filter sterilized D- or L-tryptophane or DL-tryptophane (Trp; Sigma) is added at a concentration of e.g. 0.3% by weight. The pH is adjusted to 5.5. 10 ml of the medium are poured into sterile petri dishes (10 cm diameter) and an appropriate population of Malassezia furfur (CBS 1878) is spread thereon. The substances may also be obtained in liquid medium (by omitting the agar portion).

After an incubation time of about 14 days at 30 to 37° C. the nutrient carrier is extracted with ethyl acetate and the pityriarubines are isolated by means of column chromatography and preparative high performance liquid chromatography (HPLC).

Thus the R_{f} values of the pityriarubines A, B and C, respectively, with the solvent toluene/ethyl formate/formic acid (10:5:3) on silica gel 60 plates (Merck) are e.g. approx. 0.27 (pityriarubine A), 0.14 (pityriarubine B) and 0.38 (pityriarubine C), respectively.

The retention times of these substances detected by means of HPLC in acetonitrile/water 2:3 (v/v) on a Merck-Hitachi device, equipped with a Rp18 column, 4 mm² diameter at a flow of 1 ml/min and a pressure of 140 to 160 bar, a sensitivity of 0.3 (mVolt) and a measuring frequency of the detector of 220 nm and a linear gradient of water with an increasing proportion of acetonitrile (of 0 to 100% in steps of 1%/min) are e.g. for the pityriarubines A, B and C, respectively:

Pityriarubine A: 49 min

Pityriarubine B: 48 min

Pityriarubine C: 52 min

A) Inhibition of the Protein Kinase C Activity In Vitro.

For testing the inhibitory effect on protein kinase C a commercially available protein kinase C assay kit (e.g. Calbiochem, Cat. No. 538484) may be employed. For this purpose the phosphorylation of a pseudosubstrate is measured by means of a specific antibody. The substances to be investigated are employed as solution in dimethylsulfoxid. A volume of 10 μl dimethylsulfoxid/100 μl does not interfere with the PKC dependent phosphorylation step. By means of this assay the effects on individual PKC isoforms and the inhibition thereof may be tested. The effective concentration may be then determined at a given ATP concentration.

According to this Calbiochem kit it is worked with a batch of 200 μl/well in the following manner: 13 μl of buffer solution, 26 μl of 1 mM ATP (pH 7.0), 13 μl of phosphatidyl serine (500 μg/ml), 13 μl of 20 mM CaCl₂, 65 μl of H₂O and 55 μl of H₂O, respectively, and 10 μl of inhibitor solution in DMSO are mixed, wherein 108 μl are added to an uncoated well. 12 μl of PKC solution (protein content and activity respectively of 0.1 U/12 μl) are added thereto and 100 μl thereof are added to the coated microtiter plate. After incubation for 20 min at room temperature 100 μl stop solution is added. Then washing is done five times with washing solution. Furthermore, 100 μl of biotinylated antibody/well is added and incubated for 60 min at room temperature, washed five times and incubated with 100 μl of peroxidase conjugated streptavidin/well for 60 min at room temperature. Thereafter it is washed five times and incubated with 100 μl of substrate solution/well for 3 to 10 min depending on the color intensity. Thereafter there is again a treatment with 100 μl of stop solution, and then the measurement is done in the Elisa reader at 492 nm.

The effect of the substances resides in the competitive inhibition of protein kinases, in particular of protein kinases C and other enzymes involved in signal transduction. They bind to the ATP binding site and thus disturb the binding of ATP to the enzyme. Thus they prevent the incorporation of a phosphate group into the substrate and thus the subsequent cascade of signal transduction processes. The effective concentration is 10⁻⁶ to 10⁻¹² M.

B) Antibacterial Effect Against Gram-Positive Bacteria.

Diffusion test for the evaluation of antimicrobial effects.

Müller-Hinton agar plates (Merck) were inoculated with reference strains of different bacterial species. Subsequently a mixture of crude extract and fractions thereof, respectively (each dissolved in DMSO) and 0.1 M phosphate buffer pH 7.0 (40 μl each) was dropped onto the inoculated plates and the inhibitory effect after a one day incubation at 37° C. was evaluated. As a control, a mixture of DMSO and phosphate buffer (again 40 μl each) was used and the plates were incubated for 24 h at 37° C.

The following bacterial strains were tested: Staphylococcus aureus (also MRSA), Streptococcus faecalis, Escherichia coli, Escherichia coli (+β-lactamase), Pseudomonas aeruginosa.

With the gram-positive bacteria a clear inhibition halo could be observed up to an absolutely employed amount of 1 μg.

For the preparation of pharmaceutical mixtures of the substances of the present invention, the substances may be applied in a 0.1% (w/w) dispersion in Ungt. emulsificans. According to the present invention e.g. other compositions and bases as well as in particular the combination with stabilisators, antioxidants (e.g. tocopherole), light protection agents, glucocorticosteroids and other anti-inflammatory substances, vitamin A acid and its derivatives may be used in different quantity ratios. According to the present invention, typical auxiliary agents and additives may be employed for topical preparations as further additives.

Systemically the substances may be employed in different administration forms (tablet, dragee, aerosol, suppository and the like) and/or in combination with conventional auxiliary agents and additives as well as parenterally, optionally after the preparation of water soluble derivatives and/or by addition of suitable solubilizers.

The immune modulators and/or PKC inhibitors of the present invention may be employed for preparing preparations against the following disease related skin and organ alterations:

inflammatory skin diseases;

inflammatory organ alterations and system diseases;

skin and organ alterations related to infections as well as generalized inflammatory processes such as sepsis, in particular also when bacterial causative agents are involved;

neoplastic and proliferative processes;

prophylaxis of graft rejection after organ and bone marrow transplantation.

C) Effects on the Granulocyte Burst

The preparation of granulocytes and the measurement of the superoxide release was done essentially according to Grimminger, F., K. Hattar, C. Papavassilis, B. Tenunesfeld, E. Csernok, W. L. Gross, W. Seeger and U. Sibelius, 1996. Neutrophil activation by anti-proteinase 3 antibodies in Wegener's granulomatosis: role of exogenous arachidonic acid and leukotriene B4 generation, J. Exp. Med. 184: 1567-1572.

Peripheral venous blood of healthy volunteers was collected in EDTA tubes (600 ml) and immediately processed for the isolation of polymorphonuclear neutrophils (PMN).

The EDTA anticoagulated blood was centrifuged in a Ficoll-Paque gradient (Pharmacia, Uppsala/Sweden), erythrocytes were sedimented with polyvinyl alcohol (Merck-Schuchardt, Hohenbrunn/Germany), and residual erythrocytes in the supernatant were removed by hypotonic lysis with distilled water (30 sec.). The cells were centrifuged, washed twice with phosphate buffer (298 mM) containing Ca²⁺ and Mg²⁺ (PBS) (150×g, 10 min, 4° C.) and suspended in phosphate buffer (PBS) at a final concentration of 5×10⁶/ml. Cell purity generally was >98% (Pappenheim staining), and cell viability was >96% (trypan blue exclusion).

The isolated PMNs (300 μl of the above suspension) after preincubation with 500 μl PBS for 10 min with and without the indicated inhibitor concentrations as well as with 75 μM cytochrome C with and without superoxide dismutase (SOD, 100 μg/sample), contained in 100 μl PBS by means of the calcium ionophor A23 (100 μl in PBS, final concentration of 1 μM), were stimulated to release superoxide (O₂⁻) (total volume of the batch of 1 ml). The O₂-release was measured via reduction of cytochrome C at 546 nm (10 min incubation at 37° C., thereafter stopping for 5 min in ice and centrifugation for 3 min at 13,000×g to remove cells). The same batch with addition of SOD (prevents the reduction of the cytochrome) served as a reference solution. The difference of the extinctions of both batches is a measure for the production of superoxide anions. The batches without addition of inhibitors served as a maximum control (100%), the percentage of the maximum control was determined from the extinctions of the inhibitor experiments. The inhibition curves (n=4 each) shown in the figure were obtained, which give evidence for a clear influence on the neutrophilic burst and the control events associated therewith.

The compounds according to the present invention may be employed as inflammation modulators, e.g. as a protein kinase inhibitor which e.g. reduces the superoxide release from neutrophilic granulocytes.

Besides an inhibition of PKC dependent signal transduction processes by the compounds of the invention also further signal transduction processes including cytokine release and leukotriene synthesis may be influenced.

Finally, the compounds according to the present invention may also be used for competitive binding, whereby physiological ligands will be displaced. The result thereof is a therapeutically useful net inhibition of the cellular event.

1. An inflammation modulator, in particular protein kinase inhibitor, which is an indole derivative and may be isolated from the yeast genus Malassezia, which has been supplied with tryptophane as a predominant nitrogen source, characterized in that it has the general formula (I) 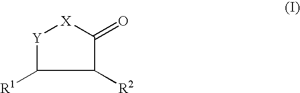
wherein:
R¹=R²=3-indole;
X═O, CH₂, a carbonyl group or a cyclic structure 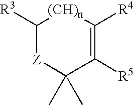
wherein n=0 or 1, R³═COOH or a ketone, Z═O or NH with the proviso that
if n=0, R⁴═OH and R⁵=3-indole;
if n=1, R⁴ taken together with R³ represents an indole ring system fused at position 2,3 and Z═NH; and
Y=O, a carbonyl group, NH or >C═N(R′)₂
wherein R′═CH₃ or C₂H₅;
with the proviso that if X= 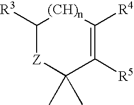
then Y is a carbonyl group,
wherein the indole ring systems may be each substituted individually or in combination with substituents, selected from the group consisting of OH, F, Cl, Br, NO₂, NH₂, COOH, HSO₃ at position 4, 5, 6, and/or 7 or form aza compounds.

2. The inflammation modulator according to claim 1, characterized in that the indole derivative has the emperical formula C₃₂H₂₂N₄O₄ and has the following structure:

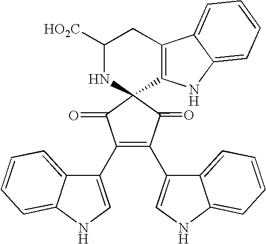

3. The inflammation modulator according to claim 1, characterized in that the indole derivative has the emperical formula C₃₂H₂₀N₄O₄ and has the following structure:

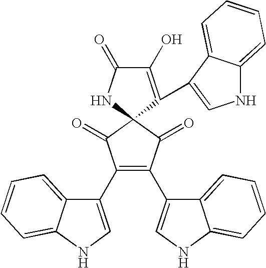

4. The inflammation modulator according to claim 1, characterized in that the indole derivative has the emperical formula C₃₂H₁₉N₃O₅ and has the following structure:

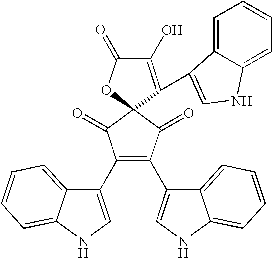

5. The inflammation modulator according to claim 1, characterized in that the indole derivative has the emperical formula C₂₀H₁₂N₂O₃ and has the following structure:

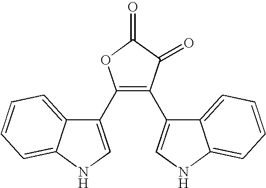

6. The inflammation modulator according to claim 1, characterized in that the indole derivative has the emperical formula C₂₀H₁₂N₃O₃ and has the following structure:

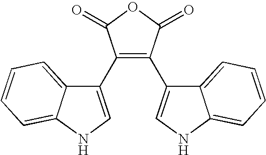

7. The inflammation modulator according to claim 1, characterized in that the indole derivative has the emperical formula C₂₁H₁₂N₂O₃ and has the following structure:

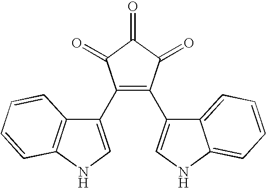

8. The use of an indole derivative according to any one of claims 1 to 7 for the preparation of a medicament for the treatment of inflammatory skin diseases, inflammatory organ alterations and systemic diseases, skin and organ alterations related to infections as well as generalized inflammation processes and neoplastic and proliferative processes, and for prophylaxis of graft rejection after organ and bone marrow transplantation.

9. The use of an indole derivative according to any one of claims 1 to 7 for the preparation of a medicament against gram-positive bacteria.

10. The use of an indole derivative according to claim 9 for the preparation of a medicament against multiresistent staphylococci.

11. The use of an indole derivative according to any one of claims 1 to 7 for the preparation of a medicament for the reduction of the superoxide release from neutrophilic granulocytes.
